(19) United States
(12) Patent Application Publication
(10) Pub. No.: US 2011/0137042 A1
(43) Pub. Date: Jun. 9, 2011

(54) Process for Synthesis of Intermediates Useful for Making Substituted Indazole and Azaindazole Compounds

(75) Inventors: Hossein RAZAVI, Danbury, CT (US); Jonathan Timothy REEVES, New Milford, CT (US); Sonia RODRIGUEZ, New Milford, CT (US)

(73) Assignee: BOEHRINGER INGELHEIM INTERNATIONAL GMBH, Ingelheim am Rhein (DE)

(21) Appl. No.: 12/957,483

(22) Filed: Dec. 1, 2010

Related U.S. Application Data
(60) Provisional application No. 61/267,538, filed on Dec. 8, 2009.

(51) Int. Cl. C07D 213/70

(52) U.S. Cl. 546/294

(57) ABSTRACT

Disclosed are processes for preparing compounds of formula (I):

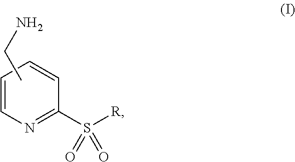

the compounds are useful as intermediates for preparing indazole and azaindazole substituted compounds.

### APPLICATION DATA

This application claims benefit to U.S. provisional application Ser. No. 61/267,538 filed Dec. 8, 2009.

### BACKGROUND OF THE INVENTION

1. Technical Field

This invention relates to novel processes for preparing compounds of the formula (I):

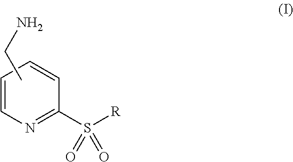

which are useful as intermediate compounds for the preparation of indazole and azaindazole substituted compounds.

2. Background Information

Indazole and azaindazole substituted compounds of formula II have been described as inhibitors of CCR1. Examples of such compounds are reported in WO 2009/134666 and WO 2010/036632. The compounds are useful for treating a variety of diseases and disorders that are mediated or sustained through the activity of CCR1 including autoimmune diseases, such as rheumatoid arthritis and multiple sclerosis.

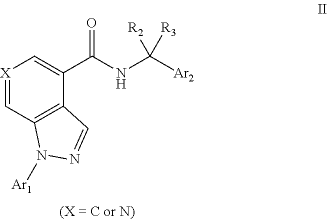

A key step in the synthesis of these compounds is the formation of the amide bond. Various methods have been reported to accomplish this. For example, as reported in the WO 2010/036632 reference, compounds of formula II described therein may be prepared by reacting (V) with an amine of the formula (VI) as shown in the Scheme:

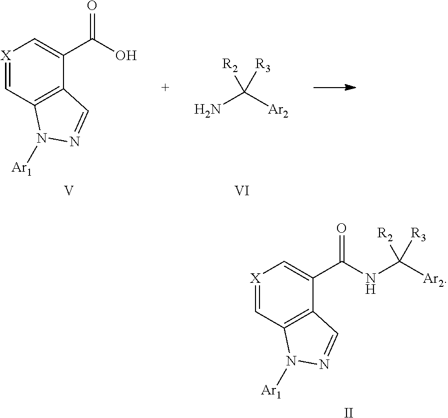

An essential intermediate in the above described synthesis of indazole and azaindazole substituted carboxamide compounds is the amine intermediate VI. The known synthesis of the amine intermediate VI involves the conversion of the cyano compound below to the corresponding amine and is done by a 2-step process involving 1) reduction with sodium borohydride/trifluoroacetic acid/zinc bromide and in-situ tert-butoxycarbonylation,

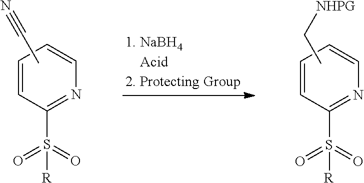

and 2) deprotection of the tert-butoxycarbonyl group using concentrated hydrochloric acid in isopropanol,

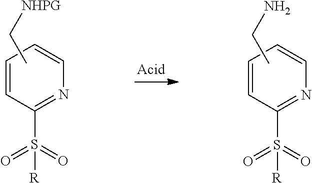

### BRIEF SUMMARY OF THE INVENTION

The synthesis of in the present invention has advantages over known processes by

1) requiring one step instead of 2 steps, thus decreasing labor costs and cycle time;

2) decreasing cost, as no Boc-anhydride, zinc bromide, NaBH₄, or TFA is required;

3) increasing safety, as it would avoid the potential for borane generation when NaBH₄/TFA is used;

4) hydrogenation can be used on industrial, commercial scale.

It is therefore an object of the invention to provide a general process with the aforementioned advantages for the preparation of amine intermediate compounds of the formula (I).

### DETAILED DESCRIPTION OF THE INVENTION

In the broadest generic embodiment, there is provided a process of making a compound of the formula (I):

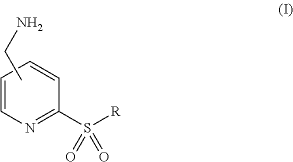

in the form of an ionic salt, comprising:

i) hydrogenating a compound of the formula (II) using a metal catalyst, preferably a Pd or Ni based catalyst, more preferably Palladium over Carbon, most preferably 10% Pd/C with water, even more preferably 10% Pd/C/50% water, with hydrogen, preferably hydrogen at pressures of 15-1000 psi, preferably 100-200 psi for 2-20 hours, preferably 7 hours, at 0-100° C., preferably 25° C., and filtration away from the catalyst followed by treatment with an acid solution or gas, preferably concentrated aqueous hydrochloric acid, the reaction is performed in a solvent chosen from an alcohol solvent, ester solvents, aqueous acids, ethers and toluene or other aromatic hydrocarbons solvents, preferably methanol, ethanol, isopropanol, or acetic acid, more preferably methanol, to provide a compound of the formula (I):

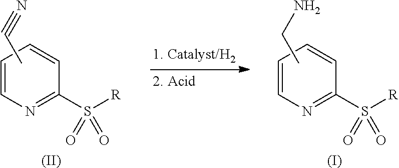

wherein R is hydrogen or C1-10 alkyl, preferably C1-5 alkyl, more preferably methyl.

In another embodiment of the invention there is provided a process of making a compound of the formula (I) according to the embodiment immediately above and wherein

the nitrile of formula (II) is on the 4 position:

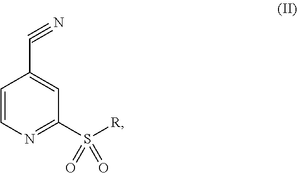

and the resulting amine group is on the 4 position of the formula (I)

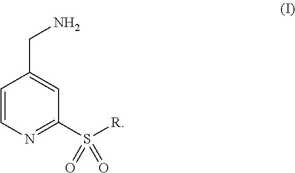

All terms as used herein in this specification, unless otherwise stated, shall be understood in their ordinary meaning as known in the art.

The term “alkyl” refers to a saturated aliphatic radical containing from one to ten carbon atoms. “Alkyl” refers to both branched and unbranched alkyl groups.

The compounds of the invention are only those which are contemplated to be ‘chemically stable’ as will be appreciated by those skilled in the art.

In order that this invention be more fully understood, the following examples are set forth. These examples are for the purpose of illustrating preferred embodiments of this invention, and are not to be construed as limiting the scope of the invention in any way.

### SYNTHETIC EXAMPLES

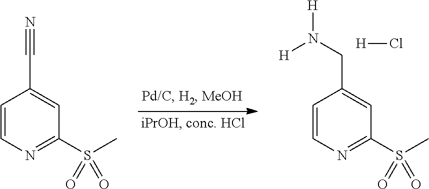

A hydrogenation vessel is charged with 2-(methanesulfonyl)-4-cyanopyridine (8.00 g, 43.9 mmol), 10 wt. % Pd/C (50% water) (800 mg, 0.377 mmol) and MeOH (48 mL). The mixture is hydrogenated under 100 psi of hydrogen at 25° C. for 7 hours. The reaction mixture is filtered to remove the catalyst, using MeOH to rinse, and the filtrate is concentrated to a volume of 24 mL. Isopropanol (48 mL) is added, followed by concentrated hydrochloric acid (4.03 mL, 48.3 mmol, 1.1 eq). The resulting slurry is stirred for 18 hours, filtered, and the resulting solid is washed with isopropanol and dried under vacuum. The product, 2-(methylsulfonyl)pyridin-4-yl)methanamine hydrochloride, is obtained as a solid (8.10 g, 82% yield) with no desulfonyl impurity by HPLC analysis, and with a residual Pd content of 46 ppm.

1. A process of making a compound of the formula (I): 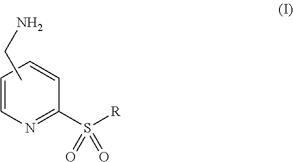
in the form of an ionic salt, comprising:
i) hydrogenating a compound of the formula (II) using a metal catalyst, with hydrogen, for 2-20 hours at 0-100 ° C., and
ii) filtering away the catalyst followed by treating with an acid solution or gas, wherein the reaction is performed in a solvent chosen from an alcohol solvent, ester solvents, aqueous acids, ethers and toluene or other aromatic hydrocarbons solvents, to provide a compound of the formula (I): 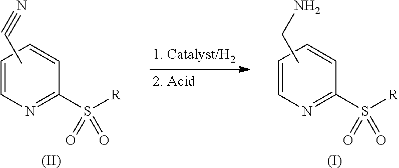
wherein R is hydrogen or C1-10 alkyl.

2. The process according to claim 1 wherein:
the metal catalyst a Pd or Ni based catalyst;
the hydrogen is at pressures of 15-1000 psi,
the time is 7 hours;
the temperature is 25° C.;
the acid is concentrated aqueous hydrochloric acid;
the solvent is chosen from methanol, ethanol, isopropanol and acetic acid;
the ionic salt is a hydrochloride.

3. The process according to claim 2 wherein:
the metal catalyst Palladium over Carbon;
the hydrogen is at pressures of 100-200 psi,
the solvent is methanol.

4. The process according to claim 3 wherein:
the metal catalyst 10% Palladium over Carbon, with water.

5. The process according to claim 4 wherein:
the metal catalyst 10% Palladium over Carbon, with 50% water.

6. The process according to any one of claims 1-5 wherein
the nitrile of formula (II) is on the 4 position: 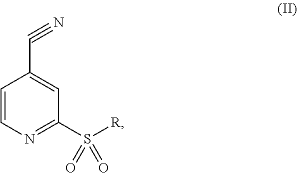
and the resulting amine group is on the 4 position of the formula (I)

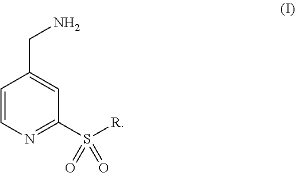

7. The process according to claim 6 wherein R is C1-5 alkyl.

8. The process according to claim 6 wherein R is methyl.
